(19) United States
(12) Patent Application Publication
(10) Pub. No.: US 2023/0085602 A1
(43) Pub. Date: Mar. 16, 2023

(54) PDAC TREATMENT REGIMEN

(71) Applicant: Swedish Orphan Biovitrum AB (publ ), Stockholm (SE)

(72) Inventors: Bent Winding, Stockholm (SE); Christina Kaiser, Stockholm (SE); Gunilla Huledal, Stockholm (SE)

(73) Assignee: Swedish Orphan Biovitrum AB (publ ), Stockholm (SE)

(21) Appl. No.: 17/759,908

(22) PCT Filed: Feb. 4, 2021
(86) PCT No.: PCT/EP2021/052613

§ 371 Date: Aug. 1, 2022

(30) Foreign Application Priority Data
Feb. 4, 2020 (EP) 20155461.5

(51) Int. Cl. A61K 38/20; A61K 31/337; A61K 31/4745; A61K 31/513; A61K 31/519; A61K 31/555; A61K 31/7068; A61K 33/243; A61K 45/06; A61P 35/00; A61K 9/00

(52) U.S. Cl. CPC A61K 38/2006; A61K 31/337; A61K 31/4745; A61K 31/513; A61K 31/519; A61K 31/555; A61K 31/7068; A61K 33/243; A61K 45/06; A61P 35/00; A61K 9/0019

(57) ABSTRACT

The human Interleukin 1 Receptor antagonist anakinra can be used in the treatment of pancreatic ductal adenocarcinoma (PDAC). The treatment involves administration of a daily dose of approximately at least 200 mg of anakinra to a patient in need thereof, for example to a patient who is undergoing a chemotherapy treatment regimen. The anakinra can be used in related treatment methods and corresponding pharmaceutical compositions.

### TECHNICAL FIELD

The present invention relates to the human Interleukin 1 Receptor antagonist anakinra for use in the treatment of pancreatic ductal adenocarcinoma (PDAC). Also, related treatment methods and pharmaceutical compositions for said use are disclosed.

### BACKGROUND

Pancreatic ductal adenocarcinoma (PDAC) is an epithelial, exocrine pancreatic malignancy, accounting for more than 80% of the malignant neoplasms of the pancreas. PDAC is rarely diagnosed in persons younger than 40 years of age, with a peak incidence of 65 to 75 years and the median age at diagnosis has been estimated to 71 years (Ryan D P et al., Pancreatic adenocarcinoma. N Engl J Med. 2014;371(11):1039-49). Symptoms of the primary malignancy usually relate to the tumor size, but can manifest as disrupted endocrine or exocrine function, asthenia, anorexia, weight loss, abdominal pain, and choluria. Due to the lack of specific signs, except for jaundice, combined with its biological aggressiveness, diagnosis is late in the disease course in 80% of cases. Approximately 50% of diagnosed patients present with metastatic disease.

PDAC is a major cause of cancer-associated mortality, with an overall 5-year survival of 4 to 7% (Adamska A et al., Pancreatic Ductal Adenocarcinoma: Current and Evolving Therapies. lnt J Mol Sci. 2017;18(7). The median overall survival for metastatic PDAC (mPDAC, also referred to as stage IV PDAC) is generally reported to be between 4 and 14 months (Cartwright T H et al., Clinical Outcomes with First-Line Chemotherapy in a Large Retrospective Study of Patients with Metastatic Pancreatic Cancer Treated in a US Community Oncology Setting. Drugs Real World Outcomes. 2018;5(3):149-59.). Even for those patients who successfully undergo tumor resection, the 5-year survival rate is at best 37%. For patients diagnosed at later stages, the 5-year survival is only 2%. Once metastasized (stage IV), PDAC prognosis is poor, and palliative chemotherapy is the main treatment option.

The lethality of PDAC stems from a lack of early detection, the nature of the malignancy itself, and, most importantly, few available therapeutic options, that in addition show limited efficacy while carrying significant toxicities. As a result, pancreatic cancer is to be considered a medical emergency (Lohr J M. Pancreatic cancer should be treated as a medical emergency. BMJ. 2014;349:g5261).

Little is known about the etiology of PDAC; however, several risk factors for development of this malignancy have been identified, including increasing age, cigarette smoking and alcohol abuse. In addition, higher rates of PDAC have been observed in patients with family history of PDAC, obesity, diabetes mellitus, chronic pancreatitis, gastric ulcers, BRCA1/BRCA2 mutation carriers, and chronic hepatitis B and C (Sclafani F et al., Management of metastatic pancreatic cancer: Current treatment options and potential new therapeutic targets. Crit Rev Oncol Hematol. 2015;95(3):318-36; Bond-Smith G et al., Pancreatic adenocarcinoma. BMJ. 2012;344:e2476; Cascetta P et al., Pancreatic Cancer and Obesity: Molecular Mechanisms of Cell Transformation and Chemoresistance. Int J Mol Sci. 2018;19(11); and Blair A B et al., BRCA1/BRCA2 Germline Mutation Carriers and Sporadic Pancreatic Ductal Adenocarcinoma. J Am Coll Surg. 2018;226(4):630-7 e1).

The incidence of pancreatic cancer in the US is 12.4 per 100 000, the majority of these cases being PDAC (Surveillance Epidemiology and End Results (SEER): Cancer stat facts: Pancreatic cancer. 2015). Although PDAC has a low incidence, it is projected to become the second most common cause of cancer death in the US by 2020 (Pokorny A M J et al., Metastatic pancreatic ductal adenocarcinoma: diagnosis and treatment with a view to the future. Intern Med J. 2018;48(6):637-44). The median overall survival for mPDAC is generally reported to be between 4 and 14 months, with around 10% of patients living more than 2 years with modern combination chemotherapy, i.e. the current standard of care (Cartwright et al., supra).

Treatment options for PDAC are limited and highly dependent on the disease stage. Treatment with chemotherapies such as gemcitabine and nab-paclitaxel (GNP), platinum compounds for BRCA1/BRCA2 mutation carriers and folfirinox (as well as variants thereof) in resected PDAC have shown significant survival improvements, however the survival continues to be low for PDAC patients.

The genetic basis of PDAC is highly complex and heterogeneous (Sclafani et al., supra). PDACs have few prevalent genetic mutations, the most commonly mutated genes being KRAS, CDKN2A, TP53 and SMAD4, all of which have proven to be difficult to target with drugs (Sclafani et al., supra; Kleeff J et al., The impact of diabetes mellitus on survival following resection and adjuvant chemotherapy for pancreatic cancer. Br J Cancer. 2016;115(7):887-94).

To summarize, PDAC is a devastating disease with poor prognosis and rising incidence. Late detection and a particularly aggressive biology are the major challenges which determine therapeutic failure. Thus, there is a large and unmet medical need for new and improved therapies for treatment of PDAC, in particular stage IV PDAC.

### SUMMARY OF THE INVENTION

It is an object of the present disclosure to provide new therapies for patients suffering from PDAC.

It is an object of the present disclosure to provide a therapeutic agent which may be used alone or in combination with other agents to treat PDAC, for example to cure PDAC or prevent or slow down PDAC disease progression. In particular, an object of the present disclosure is to provide an agent for use as an add-on therapy to a chemotherapeutic treatment regime for PDAC.

These, and other objects which are evident to the skilled person from the present disclosure, are met by the different aspects of the invention as claimed in the appended claims and as generally disclosed herein.

The present inventors have surprisingly identified that the administration of at least approximately 200 mg of the human interleukin 1 receptor antagonist anakinra leads to improved outcomes for PDAC patients. The hIL-1Ra anakinra is a recombinant, non-glycosylated form of the human IL-1Ra. The amino acid sequence of anakinra is identical to the naturally occurring protein except for the addition of an N-terminal methionine residue. Anakinra is a 153-amino acid protein with an approximate molecular weight of 17.3 kDa. Anakinra is produced by recombinant DNA technology in an E. coli expression system. Therapeutically, anakinra neutralizes the biological activity of IL-1 (IL-1α and IL-1β) by competitively inhibiting its binding to the IL-1RI.

Previous preclinical evidence provides for the benefits of reducing IL-1 activity when treating PDAC. Without being bound by theory, the present inventors have surprisingly found that the administration of at least approximately 200 mg, such as approximately 200 mg or such as at least 200 mg, of anakinra daily may be particularly efficient in reducing inflammation in the dense stroma surrounding the pancreatic tumor. In solid tumors, the malignant cells are embedded in the tumor microenvironment, which consist of stromal cells (i.e. fibroblasts and endothelial cells) and leukocytes. Cellular elements of the tumor microenvironment enable the proliferation of the malignant cells, their local invasiveness, migration to remote organs and metastasis formation. At the same time, the tumor also activates immune evasion mechanisms that suppress the anti-tumor immune responses. Thus, the majority of the tumors “hijack” the environment for their benefit to proliferate and invade host tissue. In general, inflammation and immunity against the malignant cells represent hallmarks of the tumor microenvironment, and the relationship between them determines the outcome of the malignant disease. The present inventors have realized that therapeutic approaches targeting the microenvironment should enable to tilt the balance between inflammation and anti-tumor immunity in favor of the latter in PDAC patients. Most approaches to treat cancer include therapies that boost anti-tumor responses, limit the growth of malignant cells and/or induce apoptosis (i.e. chemotherapy, irradiation), however only a limited number of approaches have been focused on targeting the tumor microenvironment. The present inventors have found that a daily dose of at least approximately 200 mg, such as approximately 200 mg or such as at least 200 mg, anakinra is particularly effective in tumor microenvironment and may improve outcomes for PDAC patients when administered alone or in combination with a chemotherapy treatment regimen.

Thus, in a first aspect of the disclosure, there is provided anakinra for use in the treatment of pancreatic ductal adenocarcinoma (PDAC), wherein said treatment comprises administration of a daily dose of at least approximately 200 mg, such as approximately 200 mg or such as at least 200 mg, such as 200 mg, anakinra to a patient in need thereof.

As used herein, the term “approximately” is to be interpreted as ±5% in relation to a numberic values. Consequently, the term approximately 200 is to be understood as 190-210. Thus, approximately 200 mg amakinra is to be understood as 190-210 mg anakinra.

It is envisioned that the administration of anakinra alone at the dose of at least approximately 200 mg, such as approximately 200 mg or such as at least 200 mg, such as 200 mg, daily would have beneficial clinical outcomes for a patient suffering from PDAC. For example, it is expected that a clinically significant reduction of carcinogenesis, tumor growth, metastasis, immunosuppression and/or angiogenesis is observed. It is also envisioned that said administration would make the tumor more susceptible to other treatments (for example by effecting the tumor microenvironment), such as chemotherapy treatment, regardless of chemotherapy type used, or irradiation treatment (also referred to as radiotherapy).

In one embodiment, there is provided anakinra for use as disclosed herein, wherein said patient is undergoing a PDAC treatment regimen, such as anotherPDAC treatment regimen, such as a chemotherapy treatment regimen and/or a radiotherapy treatment regimen.

As used herein, the term “PDAC treatment regimen” refers to a treatment received by a patient suffering from PDAC, which treatment aims at curing PDAC, or preventing or slowing down PDAC disease progression. Examples of PDAC treatment regimens include but are not limited to chemo- and/or radiotherapy. To clarify, said another PDAC treatment regimen refers to a treatment which is not administration of at least approximately 200 mg, such as approximately 200 mg or such as at least 200 mg, such as 200 mg, anakinra as disclosed herein. In the context of the present disclosure, it is to be understood that the wording “a PDAC treatment regimen” in “patient is undergoing a PDAC treatment regimen” refers to a treatment which is not the administration of at least approximately 200 mg, such as approximately 200 mg or such as at least 200 mg, such as 200 mg, anakinra as disclosed herein. In other words, the wording “a PDAC treatment regimen” in this context thus refers to another PDAC treatment, such as a chemotherapy treatment regimen and/or a radiotherapy treatment regimen.

In one embodiment, said patient is undergoing a chemotherapy treatment regimen. Optionally, said patient may undergo, for example simultaneously, an additional PDAC treatment regimen, or are regimen which mitigates adverse side effects of a PDAC treatment regimen, such as for example a regimen comprising administration of granulocyte colony stimulating factors. Without being bound by theory, it is expected that the effects of anakinra on for example, but not limited to, the tumor microenvironment, immunosuppression and/or angiogenesis may lead to an improved sensitivity of the tumor or tumors to chemotherapeutic agents. This effect is expected to occur independent of the identity of the agent as discussed in detail below. Similarly, the effects of anakinra are also envisioned to improve the sensitivity of the tumor or tumors to radiotherapy treatment.

In one embodiement of this aspect there is provided anakrinra for used as disclosed herein, wherein the daily dose of at least approximately 200 mg, such as approximately 200 mg or such as at least 200 mg, such as 200 mg, anakinra is administered at more than one separate administration occasion per day. To clarify, the daily dose of at least approximately 200 mg, such as approximately 200 mg or such as at least 200 mg, such as 200 mg, is the total dose administered during one day in other words the administration of at least approximately 200 mg, such as approximately 200 mg or such as at least 200 mg, such as 200 mg, anakinra is distributed over the course of one day.

As used herein, the term “daily dose” refers to the total dose administered during one day to a patient.

As used herein, the term “day” is to be interpreted as a continuous 24-hour period.

In one embodiment, said daily dose of at least approximately 200 mg, such as approximately 200 mg or such as at least 200 mg, such as 200 mg, anakinra is administered at least two separate administration occasions per day, such as at two separate administration occasions per day. By administering anakinra at more than one administration occasion per day it may be possible to maintain a more stable plasma concentration than if administration occurs at one administration occasion only per day. An administration regimen comprising two or more administration occasions per day may aid in maintaining a sufficiently high exposure of the tumor to anakinra throughout the day, which may beneficial and increase the effects of anakinra on the tumor or tumor(s).

As used herein, the term “administration occasion” refers to a limited time period wherein a drug is administered to a patient in need thereof. To clarify, the administration occasion encompasses the time period which is required for the administration of the entire dose of the drug which is to be administered at this time/occasion. For example, an administration occasion may be the time period required for one or several subcutaneous injections, said one or several injections representing the complete dose to be administered at the specific time/occasion. Also, an administration of a dose which requires for example a 3-hour intravenous administration is to be regarded as one administration occasion. It is to be understood that the time period of an administration occasion is significantly shorter than the period in between two administration occasions. For example, if a dose of a drug to be administered by subcutaneous injection is 100 mg, the administration of two injections of 50 mg is considered to be encompassed by the same administration occasion, provided that the time period between the administration of said two 50 mg injections is shorter than the intended period between a first administration of an entire dose of 100 mg and a second administration of an entire dose of 100 mg.

It will be appreciated that fewer number of administration occasions are generally beneficial for patient compliance and convenience of treatment, however the number of administration occasions needs to be balanced with the obtained clinical effects which may in part be dependent on maintaining a stable pharmacokinetic profile and/or level of tumor exposure to anakinra. This may for example be achieved by distributing the daily dose of anakinra over a number of administration occasions during the course of one day.

In one embodiment, said daily dose of at least approximately 200 mg, such as approximately 200 mg or such as at least 200 mg, such as 200 mg, anakinra administered on at least two separate administration occasion per day is administered as two or more essentially equal doses per day. In one particular embodiment, said daily dose of at least approximately 200 mg, such as approximately 200 mg or such as at least 200 mg, such as 200 mg, anakinra administered on at least two separate administration occasion per day is administered as two doses per day of at least approximately 100 mg anakinra each, such as two doses of approximately 100 mg anakinra each, such as two doses of 100 mg anakinra each. By dividing the daily dose of at least approximately 200 mg, such as approximately 200 mg or such as at least 200 mg, such as 200 mg, into two doses of at least approximately 100 mg anakinra each or approximately 100 mg each or at least 100 mg each or 100 mg each, without being bound by theory, it is envisioned that the effects on the tumor will be increased due to more stable (for example in terms of less fluctuating plasma concentration) therapeutically effective exposure to anakinra. For example effects may be increased on, but not limited to, the microenviroment of the tumor, immunosuppression and/or angionesis due to said more stable therapeutically effective exposure to anakinra. It is considered that it may be beneficial that the administration occasions are distrubuted, such as evenly distributed, throughout the day. It is envisioned that an even distribution of administration occassions during the day contributes to a more stable pharmacokinetic profile and ensures that any peak through fluctuations are minimized.

Thus, in one embodiment of the first aspect, there is provided anakinra for use as described herein, wheren said at least two separate administration occasions per day are at least approximately 8 hours apart. In particular, said on least two separate administration occasions per day maybe approximately 8-16 hours apart, such as approximately 9-15 hours apart, such as approximately 10-14 hours apart, such as approximately 11-13 hours apart, such as approximately 12 hours apart. In one particular embodiment said at least two separate administration occasions per day are approximately 12 hours apart.

It will be appreciated that it is important that a patient experiences as little discomfort as possible during administration of a drug. Anakinra may be administered as a subcutaneous injection or for in the form of an intravenous administration to a patient.

Thus, in one embodiment, anakinra for use as disclosed herein is provided, wherein said administration is subcutaneous administration or intravenous administration. In particular, it is convenient if the patient may be administered or may self-administer a drug in the comfort of his or her home environment.

Thus, in some embodiments as disclosed herein, the administration of anakinra is subcutaneous administration, such as subcutaneous self-administration. Alternatively, the administration may be intravenous administration. Intravenous administration may be a useful option if a patient does not tolerate or is oversensitive to subcutaneous administration.

The most widely used cancer staging system for pancreatic cancer is the one formulated by the American Joint Committee on Cancer (AJCC) together with the Union for International Cancer Control (UICC). The AJCC-UICC staging system, also referred to as the AJCC staging system, designates four main overall stages, ranging from early to advanced disease, based on TNM classification of Tumor size, spread to lymph Nodes, and Metastasis. The skilled person is familiar with the staging of PDAC. In the present disclosure that AJCC staging system is used to describe the stage of PDAC. Briefly, the PDAC stages can be summaries below and shown in Table 1. The stages are 0, I (divided into IA and IB), II (divided into IIA and IIB), III and IV and are further characterized into stage groupings based on: the extent of the tumor (T)—in other words how large the tumor is and if it has grown outside the pancreas into nearby blood vessels; the spread to nearby lymph nodes (N)—in other words if the cancer has spread to nearby lymph nodes and if so, to how many the lymph nodes has it spread; and metastasis to distant sites (M)—in other words if the cancer has spread to distant lymph nodes or distant organs such as the liver, peritoneum, lungs or bones.

TABLE 1
Stages of PDAC according to AJCC. Kindly note that stages I,
II, and III may be characterized by various TNM profiles. The
following additional categories are not listed on the table
above: TX: Main tumor cannot be assessed due to lack of information;
T0: No evidence of a primary tumor; NX: Regional lymph nodes
cannot be assessed due to lack of information.

| AJCC | Stage | |
|---|---|---|
| Stage | grouping | Stage description* |
| 0 | Tis | The cancer is confined to the top layers of |
| | N0 | pancreatic duct cells and has not invaded |
| | M0 | deeper tissues. It has not spread outside of |
| | | the pancreas. These tumors are sometimes |
| | | referred to as carcinoma in situ (Tis). |
| | | It has not spread to nearby lymph nodes (N0) |
| | | or to distant sites (M0). |
| IA | T1 | The cancer is confined to the pancreas and is |
| | N0 | no bigger than 2 cm (0.8 inch) across (T1). |
| | M0 | It has not spread to nearby lymph nodes (N0) |
| | | or to distant sites (M0). |
| IB | T2 | The cancer is confined to the pancreas and is |
| | N0 | larger than 2 cm (0.8 inch) but no more than |
| | M0 | 4 cm (1.6 inches) across (T2). |
| | | It has not spread to nearby lymph nodes (N0) |
| | | or to distant sites (M0). |
| IIA | T3 | The cancer is confined to the pancreas and is |
| | N0 | bigger than 4 cm (1.6 inches) across (T3). |
| | M0 | It has not spread to nearby lymph nodes (N0) |
| | | or to distant sites (M0). |
| IB | T2 | The cancer is confined to the pancreas and is |
| | N0 | larger than 2 cm (0.8 inch) but no more than |
| | M0 | 4 cm (1.6 inches) across (T2). |
| | | It has not spread to nearby lymph nodes (N0) |
| | | or to distant sites (M0). |
| IIA | T3 | The cancer is confined to the pancreas and is |
| | N0 | bigger than 4 cm (1.6 inches) across (T3). |
| | M0 | It has not spread to nearby lymph nodes (N0) |
| | | or to distant sites (M0). |
| IIB | T1 | The cancer is confined to the pancreas and is |
| | N1 | no bigger than 2 cm (0.8 inch) across |
| | M0 | (T1) and it has spread to no more than 3 |
| | | nearby lymph nodes (N1). |
| | | It has not spread to distant sites (M0). |
| IIB | T2 | The cancer is confined to the pancreas and is |
| | N1 | larger than 2 cm (0.8 inch) but no more than |
| | M0 | 4 cm (1.6 inches) across (T2) and it has |
| | | spread to no more than 3 nearby lymph |
| | | nodes (N1). |
| | | It has not spread to distant sites (M0). |
| IIB | T3 | The cancer is confined to the pancreas and is |
| | N1 | bigger than 4 cm (1.6 inches) across |
| | M0 | (T3) and it has spread to no more than 3 |
| | | nearby lymph nodes (N1). |
| | | It has not spread to distant sites (M0). |
| III | T1 | The cancer is confined to the pancreas and is |
| | N2 | no bigger than 2 cm (0.8 inch) across |
| | M0 | (T1) and it has spread to 4 or more nearby |
| | | lymph nodes (N2). |
| | | It has not spread to distant sites (M0). |
| III | T2 | The cancer is confined to the pancreas and is |
| | N2 | larger than 2 cm (0.8 inch) but no more than |
| | M0 | 4 cm (1.6 inches) across (T2) and it has |
| | | spread to 4 or more nearby lymph nodes |
| | | (N2). |
| | | It has not spread to distant sites (M0). |
| III | T3 | The cancer is confined to the pancreas and is |
| | N2 | bigger than 4 cm (1.6 inches) across |
| | M0 | (T3) and it has spread to 4 or more nearby |
| | | lymph nodes (N2). It has not spread to distant |
| | | sites (M0). |
| III | T4 | The cancer is growing outside the pancreas |
| | Any N | and into nearby major blood vessels (T4). |
| | M0 | The cancer may or may not have spread to |
| | | nearby lymph nodes (any N). It has not |
| | | spread to distant sites (M0). |
| IV | Any T | The cancer has spread to distant sites for |
| | Any N | example liver, peritoneum, lungs or bones |
| | M1 | (M1). It can be any size (any T) and might or |
| | | might not have spread to nearby lymph |
| | | nodes (any N). |

Additionally, for patients who may receive surgery, whether or not the tumor can be removed is an important factor. The extent of resection of tumors is classified according to: R0—all of the cancer is thought to have been removed; R1—all visible tumor was removed, but lab tests of the removed tissue show that some small areas of cancer were probably left behind; and R2—some visible tumor could not be removed. Also used are the corresponding terms resectable, borderline resectable and unresectable (either locally advanced or metastatic).

Whereas stage I, II and III PDAC may at least partially treated by surgical removal of tumor(s), stage IV PDAC is characterized by metastasis to distant sites such as the liver, peritoneum, lungs and/or bones and the surgical removal of tumor tissue, if possible, is largely insufficient for disease management. Thus, for patients who are suffering from stage IV PDAC and who have a limited number of treatment options, treatment with anakinra may be particularly important. Thus, in one embodiment, there is provided anakinra for use as disclosed herein, wherein said PDAC is stage IV PDAC. It is also comtemplated that said treatment may be useful not only a therapeutic treatment of stage IV PDAC but also a preventive treatment of stage IV PDAC. Thus, in one embodiment, said treatment is preventive treatment of stage IV PDAC.

The skilled person appreciates that most chemotherapy treatment regimens consist of days on which the chemotherapeutic agent or agents are administered alternated with days without administration of chemotherapeutic agent or agents. Different treatment regimens have different frequencies of administration of the chemotherapeutic agents and the frequencies may vary depending on if the patient is in the beginning of the treatment regimen, the middle or the end. Also, the identity of agents administered may vary between and within a chemotherapy treatment regimen. It will be appreciated that the regimens may also be adapted based on the treating physician's discretion. It is envisioned that it is beneficial that anakinra is administered to the patient independent of the stage of the chemotherapy treatment regimens. Thus, in one embodiment there is provided anakinra for use as described herein, wherein said chemotherapy treatment regimen comprises days with administration of at least one chemotherapeutic agent and days without administration of at least one chemotherapeutic agent, and wherein said anakinra is administered during days with and days without said administration of at least one chemotherapeutic agent. It is envisioned that the effects of anakinra will be beneficial to the patient independent of the identity of the chemotherapeutic agent which is administered to said patient. Hence, if a change is made in a chemotherapy treatment regimen for example in terms of dosage, frequency of administration, and/or identity of agents administered, the administration of at least approximately 200 mg, such as approximately 200 mg or such as at least 200 mg, such as 200 mg, anakinra per day may continue. A change made in chemotherapy treatment regimen may also be changing from a first line chemotherapy treatment regimen to a second or subsequent line of chemotherapy treatment regimen or changing from a second chemotherapy treatment regimen to a subsequent chemotherapy treatment regimen.

Thus, in one embodiment, the patient is undergoing a chemotherapy treatment regimen and anakinra is administered continuously independent of the identity of the chemotherapeutic agent. For example, the chemotherapy treatment regimen may change from a first line to a second line chemotherapy treatment regimen. Additionally, it is considered that anakinra may be beneficial to the patient independent of PDAC stage, and therefore if the stage of disease changes to a higher or a lower stage PDAC anakinra may still be administered to the patient. In one embodiment, the patient is undergoing a chemotherapy treatment regimen and anakinra is administered continuously independent of the PDAC stage.

As used herein, the term “administered continuously” refers to daily administration of anakinra as disclosed herein, however minor pauses in administration, for example before or after a surgical intervention, may be allowed based on treating physician's discretion.

Anakinra may be administered when patient is undergoing a first line of chemotherapy treatment regimen or a second or subsequent line of chemotherapy treatment regimen. A fist line of treatment refers to a standard treatment normally administered to a patient. When used by itself, a first line therapy is the one accepted by the profession as the best treatment. If a first line therapy does not cure or slow down or prevent progression of a disease or if the first line therapy causes severe side effects, other treatment may be added or used instead. Such treatments are referred to as second or subsequence line therapies. The presently recommended first line treatment for stage IV PDAC is combination regimen of gemcitabine and nab-paclitaxel (referred to herein as GNP regimen) as well as the folfirinox regimen, which is a combination of leucovorin, fluorouracil, irinotecan and oxaplatin. Thus, GNP and folfirinox represent non-limiting examples of first line chemotherapy treatment regimen. Non-limiting examples of second and subsequent lines of treatment for patients who received folfirinox as a first line chemotherapy treatment regimen include pembrolizumab; GNP; as well as other regimens at the discretion of the physician taking performance status and comorbidity into account. Non-limiting examples of second and subsequent lines for patients who received GNP as a first line chemotherapy treatment regimen include pembrolizumab; fluorouracil and nano-liposomal irinotecan; fluorouracil and irinotecan; fluorouracil and oxaliplatin; as well as other regimens at the discretion of the physician taking performance status and comorbidity into account.

As mentioned above, in one embodiment there is provided anakinra for use as described herein, wherein said patient is undergoing a first line of chemotherapy treatment regimen. In another embodiment, said patient is undergoing a second or subsequent line of chemotherapy treatment regimen.

In one embodiment, there is provided anakinra for use as disclosed herein, wherein said anakinra administration is continued after said patient has completed one or several chemotherapy treatment regimen(s). A patient who has completed one or several chemotherapy treatment regimen(s) may continue, directly or after a treatment free period, with a different chemotherapy treatment regimen. Alternatively, said patient may receive a different form of therapy, non-limiting examples thereof include radiotherapy and immunotherapy implementing immune checkpoint inhibitors.

A patient may also discontinue all other forms of therapy and continue to administer anakinra as disclosed herein as a monotherapy. It is also envisioned that the monotherapy may be palliative treatment.

It is also contemplated that anakinra for use as disclosed herein, may be useful for patients who have developed resistance to chemotherapy and thus are chemotherapy resistant. The terms “chemotherapy resistance” or “resistance to chemotherapy” refers to the phenomenon which occurs when cancers that have been responding to a therapy suddenly begin to grow. In other words, the cancer cells are resisting the effects of the chemotherapy. This phenomenon may be caused by mutation in the cancer cell population, changes in protein production due to gene amplification, changes in cellular transport of the chemotherapeutic agent or other reasons. Thus, in one embodiment, there is provided anakinra for use as disclosed herein, wherein said patient is chemotherapy resistant.

A number of different chemotherapeutic agent and excipients are known to be used in chemotherapy treatment. The skilled person knows that non-limiting examples of chemotherapeutic agents include alkylating agents, plant alkaloids, antitumor antibiotics, antimetabolites, topoisomerase inhibitors and miscellaneous antineoplastics and that said agents may function by affecting different aspects of tumor growth, cell cycle or division or other. For example gemcitabine and fluorouracil are pyrimidine antagonist and thus are antimetabolic agents; ironotecan is a topoisomerase I inhibitor; and oxaplatin is a metal salt which is a alkylating agent. Non-limiting examples of agents that can be included in a chemotherapy treatment regimen include actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, fluorouracil, gemcitabine, hydroxyurea, idarubicin, imatinib, irinotecan, nanoliposomal irinotecan, leucovorin, mechlorethamine, mercaptopurine, methotrexate, mitoxantrone, oxaliplatin, paclitaxel, nab-paclitaxel, pemetrexed, pembrolizumab, platinum-based antineoplastic agents, teniposide, tioguanine, topotecan, valrubicin, vemurafenib, vinblastine, vincristine and vindesine.

Thus, in one embodiment of anakinra for use as disclosed herein, said chemotherapy treatment regimen comprises administration of therapeutically effective dose of at least one agent selected for the group consisting of actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, fluorouracil, gemcitabine, hydroxyurea, idarubicin, imatinib, irinotecan, nanoliposomal irinotecan, leucovorin, mechlorethamine, mercaptopurine, methotrexate, mitoxantrone, oxaliplatin, paclitaxel, nab-paclitaxel, pemetrexed, pembrolizumab, platinum-based antineoplastic agents, teniposide, tioguanine, topotecan, valrubicin, vemurafenib, vinblastine, vincristine and vindesine, such as the group consisting of cisplatin, fluorouracil, gemcitabine, irinotecan, nanoliposomal irinotecan, leucovorin, nab-paclitaxel, oxaliplatin, pembrolizumab; such as the group consisting of leucovorin, gemcitabine, nab-paclitaxel, cisplatin, fluorouracil, oxaliplatin and irinotecan; such as the group consisting of gemcitabine, nab-paclitaxel, cisplatin, fluorouracil, oxaliplatin and irinotecan.

Particular chemotherapy treatment regimens may comprise a combination of agents which may be administered simultaneously or sequentially immediately after each other or at different occasions. Non-limiting examples of such chemotherapy treatment regimens include regimens comprising administration of leucovorin, fluorouracil, irinotecan and oxaplatin; regimens comprising administration of gemcitabine and nab-paclitaxel; regimens comprising administration of pembrolizumab; regimens comprising administration of fluorouracil and oxaplatin; regimens comprising administration of fluorouracil and irinotecan; and regimens comprising administration of fluorouracil and nanoliposomal irinotecan.

Thus, in one embodiment of anakinra for use as disclosed herein, said chemotherapy treatment regimen is selected from the group consisting of regimens a), b), c), d), e) and f), such as the group consisting of regimens a) and b), wherein

regimen a) is administration of leucovorin, fluorouracil, irinotecan and oxaplatin;
regimen b) is administration of gemcitabine and nab-paclitaxel;
regimen c) is administration of pembrolizumab;
regimen d) is administration of fluorouracil and oxaplatin;
regimen e) is administration of fluorouracil and irinotecan; and
regimen f) is administration of fluorouracil and nanoliposomal irinotecan.

In one embodiment, said regimen comprises administration of gemcitabine or nab-paclitaxel. In a particular embodiment, said regimen comprises administration of gemcitabine and nab-paclitaxel. A chemotherapy treatment regimen is often repetitive such that the same events, for example administrations, return with a specific periodicity during the duration of a treatment. For example, a cycle of 28 days may be employed. In one embodiment, wherein gemcitabine and nab-paclitaxel are administered to the patient, said administration of gemcitabine and nab-paclitaxel is on days 1, 8 and 15 of each 28-day cycle. To clarity, a subsequent cycle may start immediately after the completion of a previous cycle or alternatively a previous cyle may be followed by a pause in treatment for a subsequent cycle is initiated at the physician's discretion. In one embodiment, said administration of gemcitabine and nab-paclitaxel is intravenous administration of approximately 1000 mg/m² gemcitabine and approximately 125 mg/m²nab-paclitaxel.

In another embodiment, said regimen comprises administration of leucovorin, fluorouracil, irinotecan or oxaplatin. In one embodiment, said regimen comprises administration of leucovorin, fluorouracil, irinotecan and oxaplatin.

First line chemotherapy for state IV PDAC comprises administration of gemcitabine and nab-paclitaxel or administration of leucovorin, fluorouracil, irinotecan and oxaplatin. This in one embodiment of anakinra for use as disclosed herein, said PDAC is stage IV PDAC.

Optionally a regimen may also comprise administration of human granulocyte colony stimulating factor (G-CSF) such as filgrastim or pegfilgrastim.

In the second aspect of the present disclosure, there is provided a related method of treatment of pancreatic ductal adenocarcinoma (PDAC), wherein said treatment comprises administering as therapeutically effective dose of at least approximately 200 mg, such as approximately 200 mg or such as at least 200 mg, such as 200 mg anakinra per day to a patient in need thereof. The skilled person will appreciate the details discussed in relation to the first aspect disclosed herein are equally relevant for this second aspect and for the sake of brevity will not be repeated in full but only mentioned briefly.

In one embodiment, there is provided a method of treatment of PDAC, wherein said patient is undergoing a PDAC treatment regimen, such as another PDAC treatment regimen, such as a chemotherapy treatment regimen and/or a radiotherapy treatment regimen. As explained in the context of the first aspect, it is to be understood that the wording “a PDAC treatment regimen” in “patient is undergoing a PDAC treatment regimen” refers to a treatment which is not the administration of at least approximately 200 mg, such as approximately 200 mg or such as at least 200 mg, such as 200 mg, anakinra as disclosed herein. In other words, the wording “a PDAC treatment regimen” in this context thus refers to another PDAC treatment, such as a chemotherapy treatment regimen and/or a radiotherapy treatment regimen.

In one embodiment, said patient is undergoing a chemotherapy treament regimen. Optionally, said patient may undergo, for example simultaneously, an additional PDAC treatment regimen, or are regimen which mitigates adverse side effects of a PDAC treatment regimen, such as for example a regimen comprising administration of granulocyte colony stimulating factors.

As discussed in the context of the first aspect, it may be beneficial that the at least approximately 200 mg, such as approximately 200 mg or such as at least 200 mg, such as 200 mg dose of anakinra is administered on more than one administration occasion. Thus, in one embodiment of said method, the daily dose of at least approximately 200 mg, such as approximately 200 mg or such as at least 200 mg, such as 200 mg, anakinra is administered on more than one separate administration occasion per day. In one particular embodiment, the daily dose of at least approximately 200 mg, such as approximately 200 mg or such as at least 200 mg, such as 200 mg, anakinra is administered on at least two separate administration occasions per day, such as on two separate administration occasions per day. In one embodiment, the dose of at least approximately 200 mg, such as approximately 200 mg or such as at least 200 mg, such as 200 mg anakinra administered on at least two separate administration occasion per day is administered as two or more essentially equal doses per day. In a particular embodiment, the daily dose of at least approximately 200 mg, such as approximately 200 mg or such as at least 200 mg, such as 200 mg, anakinra administered on at least two separate administration occasion per day is administered as two doses per day of approximately 100 mg anakinra each. In particular, the daily dose of at least approximately 200 mg, such as approximately 200 mg or such as at least 200 mg, such as 200 mg, anakinra administered on at least two separate administration occasion per day is administered as two doses of at least approximately 100 mg anakinra each, such as two doses of approximately 100 mg anakinra each or two dose of at least 100 mg each, such as two doses of 100 mg anakinra each.

As discussed in relation to the first aspect, it will be appreciated that fewer number of administration occasions are generally beneficial for patient compliance and convenience of treatment, however the number of administration occasions needs to be balanced with the potentially obtained clinical effects of for example maintain a more stable pharmacokinetic profile and/or level of tumor exposure to anakinra. Furthermore, an even distribution of administration occassions during the day is envisioned to contribute to a more stable pharmacokinetic profile and to ensure that any peak through fluctuations are minimized.

Thus, in one embodiment of the method as disclosed herein, said at least two separate administration occasions per day are at least approximately 8 hours apart. For example, said two separate administration occasions per day may be at approximately 8-16 hours apart, such as approximately 9-15 hours apart, such as approximately 10-14 hours apart, such as approximately 11-13 hours apart, such as approximately 12 hours apart. In one particular embodiment, said at least two separate administration occasions per day are approximately 12 hours apart.

Of importance is that the route of administration causes as little discomfort to the patient as possible, such as for example subcutaneous administration which may be for example be performed by the patient itself. For patients who do not tolerate subcutaneous administration other administration routes may be considered. In one embodiment of the method as disclosed herein, said administration is subcutaneous administration or intravenous administration. In particular embodiments, said administration is subcutaneous administration, such as subcutaneous self-administration. In other embodiments, said administration is intravenous administration.

As explained in the context of the first aspect, PDAC may be staged according to the AJCC staging system. In one embodiment said PDAC is stage IV PDAC.

In one embodiment, there is provided a method as disclosed herein, wherein said treatment is preventive treatment of stage IV PDAC.

As discussed in the context of the first aspect, most chemotherapy treatment regimens consist of days on which the chemotherapeutic agent or agents are administered alternated with days without administration of the same or other chemotherapeutic agent(s).

In one embodiment of the second aspect as disclosed herein, there is provided a method of treatment of PDAC, wherein said chemotherapy treatment regimen comprises days with administration of at least one chemotherapeutic agent and days without administration of at least one chemotherapeutic agent, and wherein said anakinra is administered during days with and without said administration of at least one chemotherapeutic agent. In one embodiment, there is provided a method wherein the patient is undergoing a chemotherapy treatment regimen and wherein anakinra is administered continuously independent of the PDAC stage and/or identity of the chemotherapeutic agent. Anakinra may be administered when patient is undergoing a first line of chemotherapy treatment regimen or a second or subsequent line of chemotherapy treatment regimen as discussed in the context of the first aspect. Thus, in one embodiment, the patient is undergoing a first line of chemotherapy treatment regimen. In another embodiment, the patient is undergoing a second or subsequent line of chemotherapy treatment regimen.

In one particular embodiment, said anakinra administration is continued after said patient has completed a chemotherapy treatment regimen.

It may be beneficial to administer anakinra to a patient who is chemotherapy resistant as discussed above. Thus, in one embodiment there is provided a method of treatment of PDAC as disclosed herein, wherein said patient is chemotherapy resistant.

The skilled person is familiar with different types of chemotherapeutic agents and excipients which may be used in a chemotherapy treatment regimen. In one embodiment of the method as disclosed herein, said chemotherapy treatment regimen comprises administration of therapeutically effective dose of at least one agent selected for the group consisting of actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, nanoliposomal irinotecan, leucovorin, mechlorethamine, mercaptopurine, methotrexate, mitoxantrone, oxaliplatin, paclitaxel, nab-paclitaxel, pemetrexed, pembrolizumab, platinum-based antineoplastic agents, teniposide, tioguanine, topotecan, valrubicin, vemurafenib, vinblastine, vincristine and vindesine; such as the group consisting of cisplatin, fluorouracil, gemcitabine, irinotecan, nanoliposomal irinotecan, leucovorin, nab-paclitaxel, oxaliplatin, pembrolizumab; such as the group consisting of leucovorin, gemcitabine, nab-paclitaxel, cisplatin, fluorouracil, oxaliplatin and irinotecan; such as the group consisting of gemcitabine, nab-paclitaxel, cisplatin, fluorouracil, oxaliplatin and irinotecan.

In particular embodiments, said chemotherapy treatment regimen is selected from the group consisting of regimens a), b), c), d), e) and f), such as the group consisting of regimens a) and b),

wherein
regimen a) is administration of leucovorin, fluorouracil, irinotecan and oxaplatin;
regimen b) is administration of gemcitabine and nab-paclitaxel;
regimen c) is administration of pembrolizumab;
regimen d) is administration of fluorouracil and oxaplatin;
regimen e) is administration of fluorouracil and irinotecan; and
regimen f) is administration of fluorouracil and nanoliposomal irinotecan.

In one embodiment, said regimen comprises administration of gemcitabine or nab-paclitaxel. In a particular embodiment, said regimen comprises administration of gemcitabine and nab-paclitaxel. A chemotherapy treatment regimen is often repetitive such that the same events, for example administrations, return with a specific periodicity during the duration of a treatment. In one embodiment there is provided a method as disclosed herein, wherein gemcitabine and nab-paclitaxel are administered to the patient, and wherein said administration of gemcitabine and nab-paclitaxel is on days 1, 8 and 15 of each 28-day cycle. In one embodiment, said administration of gemcitabine and nab-paclitaxel is intravenous administration of approximately 1000 mg/m²gemcitabine and approximately 125 mg/m²nab-paclitaxel. In another embodiment of said method, said regimen comprises administration of leucovorin, fluorouracil, irinotecan or oxaplatin. In one embodiment, said regimen comprises administration of leucovorin, fluorouracil, irinotecan and oxaplatin. Said regimens may be useful for the treatment of stage IV PDAC. Thus, in one particular embodiment of said method, said PDAC is stage IV PDAC.

Optionally the regimen may also comprise administration of human granulocyte colony stimulating factor (G-CSF) such as filgrastim or pegfilgrastim.

In a third aspect of the present disclosure, there is provided a use of anakinra for the manufacture of a medicament for the treatment of PDAC, wherein said treatment comprises administration of a daily dose of at least approximately 200 mg, such as approximately 200 mg or such as at least 200 mg, such as 200 mg, of anakinra to a patient in need thereof, such as wherein said treatment comprises administration of two doses per day of at least approximately 100 mg anakinra each, such as two doses of approximately 100 mg anakinra each, such as two doses of 100 mg anakinra each, to a patient in need thereof. In one embodiment, there is provided the use of anakinra for the manufacture of a medicament for the treatment of PDAC as described herein, wherein said patient is undergoing a chemotherapy treatment regimen, such as a chemotherapy treatment regimen as defined herein. Embodiments of this aspect equal the embodiments described above for the first aspect related anakinra for use in treatment of PDAC and will not be repeated here for the sake of brevity.

The term “pharmaceutical composition” is used in its widest sense, encompassing all pharmaceutically applicable compositions containing at least one active substance, and optional carriers, adjuvants, constituents etc. The term “pharmaceutical composition” also encompasses a composition comprising the active substance in the form of derivate or a prodrug, such as a pharmaceutically acceptable salt, sulphate and/or ester of said active substance. The active substance may be anakinra. The manufacture of pharmaceutical compositions for different routes of administration falls within the capabilities of a person skilled in galenical chemistry.

In a fourth aspect of the present disclosure, there is provided a pharmaceutical composition comprising anakinra for use as described herein and at least one pharmaceutically acceptable excipient, diluent and/or carrier. In one embodiment, said pharmaceutical composition comprising anakinra for use as disclosed herein is for use as an add-on therapy to another PDAC treatment regimen, such as a radiotherapy and/or chemotherapy treatment regimen.

It is to be understood that the terminology employed herein is used for the purpose of describing particular embodiments only and is not intended to be limiting, since the scope of the present invention will be limited only by the appended claims and equivalents thereof. Embodiments of this aspect equal the embodiments described above for the first aspect related anakinra for use in treatment of PDAC and will not be repeated here for the sake of brevity.

In particular, it is noted that, as used in this specification and the appended claims, the singular forms “a”, “an”, and “the” also include plural referents unless the context clearly dictates otherwise.

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1 is a schematical representation of the clinical study according to Example 1. Abbrevations in the figure are as follows: GNP: Gemcitabine+Nab-Paclitaxel.

### EXAMPLES

### Summary

The present Examples describe a clinical study that aims to assess the efficacy and safety of the hIL-1R antagonist anakinra as add-on to chemotherapy in the treatment of PDAC in patients. The present inventors have established a method of treatment comprising administering anakinra, which may be administered as a monotherapy or be administered in combination with a chemotherapeutic treatment regimen to a patient in need thereof. Table 2 shows the events schedule for the study according to Example 1.

TABLE 2
Schedule of events.

| | | | | | | Follow-up period | |
|---|---|---|---|---|---|---|---|
| | | | | | | 6 months | |
| | Treatment period | | | | | End of IMP | |
| | | | | Visit 4 | | visit | Phone |
| | Visit 1¹ | Visit 2 | Visit 3 | and onwards | | 30 days | call² |
| | Baseline | Day 15 | Day 28 | Every | Progression | after last | Every |
| Assessments | (Day 1) | (Week 2) | (Week 4) | 28 days | visit | intake of IMP | 4 weeks |
| Informed consent | X | | | | | | |
| Eligibility criteria | X | | | | | | |
| Medical and surgical | X | | | | | | |
| history, and | | | | | | | |
| demographics | | | | | | | |
| Physical examination | X | | X | X | X | X | |
| incl. neuro status | | | | | | | |
| Vital signs (BP, pulse, | X | X | X | X | X | X | |
| temperature, height, | | | | | | | |
| weight) assessment | | | | | | | |
| ECG | X | | | | | | |
| CT/MRI scan | X | | | X | X | | |
| Prior and concomitant | X | X | X | X | X | X | |
| medication | | | | | | | |
| Laboratory safety | X | X | X | X | X | X | |
| assessment, local³ | | | | | | | |
| Laboratory safety | X | X | X | X | X | X | |
| assessment, central^{3, 4} | | | | | | | |
| Randomization | X | | | | | | |
| Patient/caregiver IMP | X | | | | | | |
| injection training⁵ | | | | | | | |
| IMP administration | X | | | | | | |
| training | | | | | | | |
| IMP treatment stop | | | | | X⁶ | | |
| GNP chemotherapy | [symbol] | | | | | | |
| administration | | | | | | | |
| QoL EQORTC-QLQ- | X | | X | X | X | (X) | |
| C30 | | | | | | | |
| Adverse events | X | X | X | X | X | X | |
| SAEs⁷ | X | X | X | X | X | X | X |
| IMP tracking | X | | X | X | X | | |
| Survival status | | X | X | X | X | X | X |

¹Baseline visit will be performed during Day 1, possibility to pre-screen patients on Day −1;
²Patients will be followed for SAEs and death events every 4 weeks up to maximum 6 months after date of confirmed progression;
³Including CA19-9;
⁴Including biomarkers;
⁵To be administered at study site first time, then at home during the study treatment period (until progression);
⁶IMP treatment will be stopped the same day as the patient has a confirmed disease progression according to the Investigator;
⁷From signing of informed consent. After the follow-up visit only SAEs considered by the investigator to be related to study drug will be collected.

Abbreviations and definition of terms used herein:

| Abbreviation | Definition |
|---|---|
| 5FU | fluorouracil |
| AE | Adverse event |
| BP | Blood pressure |
| CA | Carbohydrate antigen |
| CA 19-9 | CA 19-9 is a tumor marker that may be helpful in |
| | pancreatic cancer. A drop in the CA 19-9 level after |
| | surgery (compared to the level before surgery) and |
| | low levels of CA 19-9 after pancreas surgery tend |
| | to predict a better prognosis. |
| CI | Confidence interval |
| CT | Computerized tomography |
| ECOG PS | A scale and criteria used by doctors and researchers |
| | to assess how a patient's disease is progressing. |
| E. coli | Escherichia coli |
| EORTC | Questionnaire developed to assess the quality of |
| QLQ-C30 | life of cancer patients. |
| FOLFIRINOX | A combination of cancer drugs that includes: |
| | leucovorin/folinic acid, fluorouracil, irinotecan, |
| | and oxaliplatin |
| G-CSF | Granulocyte colony stimulating factor |
| GFR | Glomerular Filtration Rate |
| GNP | Gemcitabine and nab-paclitaxel |
| HR | Hazard Ratio |
| HER2 | Human epidermal growth factor receptor 2 |
| HgBA1c | Hemoglobin A1c |
| DSMBData | Safety Monitoring Board |
| IL-1 | Interleukin-1 |
| IL-1α | Interleukin 1 alpha |
| IL-1β | Interleukin 1 beta |
| IL-1R | Interleukin-1 receptor |
| IL-1RI | Interleukin-1 receptor type I |
| IL-1Ra | Interleukin-1 receptor antagonist |
| IMP | Investigational medical product |
| NAB | Nanoparticle albumin-bound |
| mPDAC | Metastatic pancreatic ductal adenocarcinoma, also |
| | referred to as stage IV PDAC. Terms mPDAC and stage |
| | IV PDAC are used herein interchangeably. |
| MRI | Magnetic resonance imaging |
| OS | Overall survival |
| PFS | Progression free survival |
| RECIST | Standard way to measure how well a cancer patient |
| | responds to treatment |
| SAE | Serious adverse event |
| SD | Standard deviation |
| SoC | Standard of care |
| TNM | Diagnosis classification criteria. T: describes the |
| | size of the main tumor and whether it grows outside |
| | of the pancreas or in neighboring organs; N: describes |
| | extension to lymph nodes; and M: describes if the |
| | tumor has extended to distant organs |
| ULN | Upper limit of normal |
| QoL | Quality of life |

### Example 1

### Clinical Study—Stage IV PDAC

The present Example relates to a randomized, double-blind, placebo-controlled, multicenter phase 2 study of anakinra as add-on to chemotherapy in the treatment of metastatic pancreatic ductal adenocarcinoma (mPDAC also referred to herein as stage IV PDAC). The main aim of the study is to evaluate the efficacy and safety of anakinra as add-on to chemotherapy in the treatment of mPDAC in adult, chemotherapy treatment-naïve patients. The primary objective is to demonstrate prolonged overall survival in patients with mPDAC receiving treatment with anakinra versus placebo, as add-on to chemotherapy. The primary endpoint is overall survival defined as the time from the date of randomization until death due to any cause. Furthermore, the study aims to demonstrate prolonged progression free survival in patients with mPDAC receiving treatment with anakinra versus placebo, as add-on to chemotherapy and to demonstrate efficacy of anakinra versus placebo, as add-on to chemotherapy in improving quality of life in patients with mPDAC.

Additionally, of interest is to evaluate early onset of efficacy of anakinra versus placebo, as add-on to chemotherapy in the treatment of mPDAC; to evaluate sustained efficacy of anakinra versus placebo, as add-on to chemotherapy in the treatment of mPDAC; to evaluate the effect of anakinra versus placebo, as add-on to chemotherapy on different aspects of quality of life (QoL) in patients with mPDAC; as well as to evaluate safety of anakinra as add-on to chemotherapy.

### Example 1a

### Study Design and Study Population

In Example 1a the design of the clinical study in described as well as the criteria relating to the study population.

Study design: A randomized, double-blind, placebo-controlled, multicenter phase 2 study of anakinra as add-on to chemotherapy in the treatment of mPDAC (stage IV PDAC) is conducted. Patients included are diagnosed with stage IV PDAC and are chemotherapy treatment-naïve and are histologically or cytologically confirmed to have mPDAC and exhibit acceptable bone marrow, liver and kidney status.

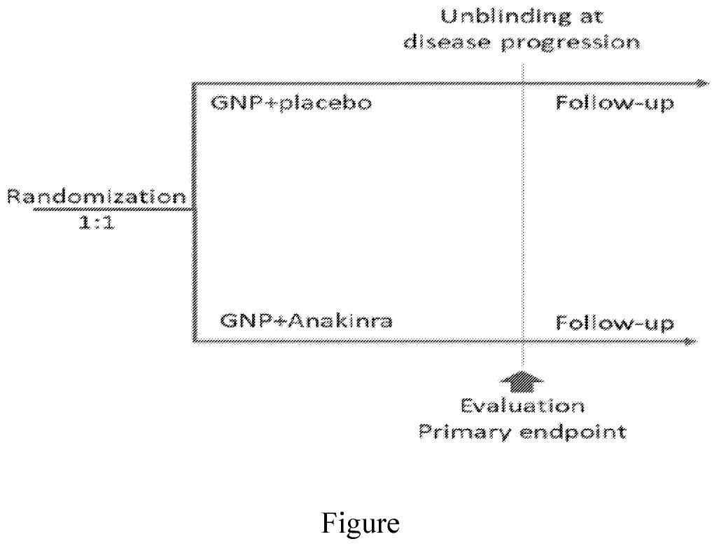

The study consists of a treatment period and a posttreatment follow-up period as illustrated in FIG. 1. Chemotherapy treatment-naïve patients with a confirmed diagnose of mPDAC who are suitable for a GNP (gemcitabine and nab-paclitaxel) regimen in the first line setting will be randomized at the baseline visit to either GNP+anakinra or GNP+placebo in a 1:1 ratio. Approximately 222 patients will be randomized in a 1:1 ratio to GNP+anakinra or GNP plus matching placebo. The total number of events that needs to be observed is 154.The randomization will be stratified at baseline by ECOG PS 0 (Yes; No); presence of liver metastasis (Yes; No) and elevated CA 19-9 levels (Yes; No).

The median PFS in the control group is assumed to be 5.5 months. If a median PFS in the placebo arm of 5.5 months and a median PFS of 7.5 months in the anakinra group are assumed, these assumptions will result in a HR of 0.71. To ensure a power of 80% in demonstrating efficacy if the true HR is 0.71, using a 1-sided alpha-level of 0.1, 154 death events need to be observed.

The enrolment period is exptected to be 12 months and final analysis is performed when the 154 events have occurred (approx. at 18 months). The study period is expected to be 24 months (the study will end 6 months after the date for Final analysis).

Anakinra (and matching placebo) will be given every calendar day as 2 subcutaneous injections, once in the morning and once in the evening, with a total daily anakinra dose of 200 mg. Anakinra, also referred to herein as investigational medical product (IMP), will be administered until disease progression.

Safety will be monitored throughout the study and three interim safety evaluations will be performed.

A Data Safety Monitoring Board (DSMB) will evaluate both safety data during the study and will keep the blinding towards the rest of the study team and sponsor.

Treatment period: Patients will start IMP treatment at Baseline and continue on IMP throughout the study. During the first month, patients will have study visits at the clinic every second week for assessments of vital signs, laboratory safety status, physical exam and QoL. Thereafter study visits will take place every 28 days and include the same assessments as above. A CT or MRI scan will be performed every 6 weeks. Table 2 gives an overview of the planned study visits.

Follow up period: Once the patient has a confirmed disease progression according to the Investigator, the treatment with anakinra will stop and patients will be unblinded and enter the Follow-up period. The Follow-up period comprises of an End of IMP visit approximately 30 days after last intake of IMP, where assessments on vital signs, laboratory status, adverse events, CT/MRI scan and physical examination including neurology status will occur. After that, patients will be followed for survival by phone calls every 4 weeks until 6 months after evaluation of primary endpoint.

Safety Analysis: Safety is evaluated according to Example 1c.

Study Population: The following inclusion and exclusion criteria apply:

Main inclusion criteria: A patient will be eligible for inclusion in this study if he or she meets all of the following criteria: 1. Histologically or cytologically confirmed diagnosis of mPDAC within 6 weeks before randomization; 2. Coexisting extra pancreatic distant metastasis; 3. Chemotherapy treatment-naïve patients suitable for a GNP treatment regimen in a first line setting; 4. Age ≥18 years old; 5. Life expectancy at least 3 months according to Investigator; 6. Measurable primary tumor in pancreas on imaging study at the time of diagnosis, according to the RECIST criteria, version 1.1; and 7. Signed informed consent.

Main exclusion criteria: A patient will be ineligible for inclusion in this study if he or she meets any of the following criteria: 1. Previous history of systemic chemotherapy; 2. History of malignancy, other than the current, within the past 5 years. Exceptions are basal cell skin cancer, carcinoma-in-situ of the cervix, and low-risk prostate cancer after curative therapy. 3. Existence of life-threatening co-morbidity; 4.Poor performance state (ECOG ≥2); 5. Severe bone marrow suppression (neutrophil count <1,500/mm3, hemoglobin <9 g/dL, platelet count <75,000/mm3); 6. Suspected severe liver dysfunction (Total bilirubin or prothrombin time >1.5 times upper reference limit); 7. Chronic kidney disease stages 4 and 5 (i.e. estimated GFR <30/ml/min/1.73 m²); 8. Pre-existence of grade 2 peripheral sensory neuropathy; 9. Existence of brain metastasis or meningeal carcinomatosis; 10. Known hypersensitivity to Escherichia coli-derived proteins, anakinra or any components of the product; 11. Exposure to an IL-1 or an IL-6 inhibitor within last 24 months; 12. Other severe and/or uncontrolled medical condition or other condition that according to the investigator could affect the participation of the patient in the study; 13. Known or suspected infection of hepatitis B, hepatitis C, tuberculosis, or HIV infection; 14. Pregnant or lactating women; 15. Any medical condition which in the opinion of the investigator makes the subject unsuitable for inclusion; 16. Enrollment in another concurrent clinical interventional study, or intake of an investigational drug, within three months prior to inclusion in this study; and 17. Foreseeable inability to cooperate with given instructions or study procedures.

Visit schedule and assessments: Patients are assessed at scheduled visits as shown in Table 2. A brief summary is as follows: Documentation and measurement of target and non-target tumor lesions by means of spiral CT or MRI is performed at baseline. Every 6 weeks after baseline, the tumor response is evaluated by means of spiral CT or MRI with the use of RECIST, version 1.1.

Safety is monitored by means of assessments by the investigators of serious and non-serious adverse events, laboratory testing performed at a central laboratory, dose modifications, dose delays, changes in type of chemotherapy regimen, and premature discontinuations of the study drug. Use of granulocyte colony stimulating factor (G-CSF) is also monitored.

Patients are followed for survival until death, patient withdrawal, or study closure.

Evaluation of patient reported outcomes by means of the questionnaire EORTC QLQ-C30 (a questionnaire developed to assess the quality of file of cancer patients) is performed at baseline and every 28 days thereafter. Serial measurements of cancer associated antigen 19-9 (CA19-9) and other biomarkers is also performed at baseline and every 28 days thereafter.

### Example 1b

### Inventive Treatment, Dose and Mode of Administration

Example 1b discloses the treatment administered and evaluated in the present clinical study, including the doses administered and treatment duration.

Treatment period first line: Patients in the treatment group (referred to as Arm A) are administered gemcitabine and nab-paclitaxel together with a daily dose 200 mg anakinra, administered as 100 mg b.i.d.

Anakinra, 100 mg subcutaneous (s.c.) injection, is administered twice daily and is given continuously, both during administration of chemotherapy and during the periods when chemotherapy is not given. Intravenous nab-paclitaxel (125 mg/m²) and gemcitabine (1000 mg/m²) is administered on days 1, 8 and 15 of each 28-day cycle. Gemcitabine is administered immediately after nab-paclitaxel.

Patient in the control group (referred to as Arm B) are administered gemcitabine and nab-paclitaxel together with a daily dose of placebo, administered mg b.i.d.

Placebo is administered twice daily by s.c. injection, and is given continuously, both during administration of chemotherapy and during the periods when chemotherapy is not given. Intravenous nab-paclitaxel (125 mg/m²) and gemcitabine (1000 mg/m²) is administered on days 1, 8 and 15 of each 28-day cycle. Gemcitabine is administered immediately after nab-paclitaxel.

Duration of treatment: Anakinra treatment starts on the first day of GNP administration. Anakinra is administered continuously until disease progression. Treatment with anakinra will be discontinued if intolerable toxicity is observed, if the patient withdraws from the study, or at the Investigator's discretion.

Treatment with GNP will discontinue if disease progression or intolerable toxicity is observed, if the patient withdraws from the study, or at the Investigator's discretion.

### Example 1c

### Safety and Efficacy Analyses

Example 1c describes the analyses of safety and efficacy as well as statistical methodology employed in the study.

Safety Analysis: Safety is evaluated by 3 predefined safety analyses will be performed by the Data Safety Monitoring Board (DSMB). The DSMB will evaluate safety data during the study and will keep the blinding towards the rest of the study team and sponsor.

The first safety analysis will be performed when 12 patients (approximately 6 patients on anakinra and 6 on placebo) have completed one GNP treatment cycle.

A second safety analysis will be performed when 40 patients (approximately 20 patients on anakinra and 20 on placebo) have completed two chemotherapy cycles.

A third safety analysis will be performed when 100 patients (approximately 50 patients on anakinra and 50 on placebo) have completed two chemotherapy cycles.

Additional evaluations of safety will be performed if required.

### Study Objectives and Statistical Methods

Primary endpoint: As mentioned above, the primary objective is to demonstrate prolonged progression free survival in patients with mPDAC receiving treatment with anakinra versus placebo, as add-on to GNP. The primary endpoint is progression free survival defined as the time from the date of randomization to disease progression, or death due to any cause, whatever happens first. The primary endpoint is progression free survival defined as the time from the date of randomization to disease progression, assessed by Investigator in accordance with RECIST 1.1, or death due to any cause, whatever happens first. Any subject not known to have progressed at the time of analysis will be censored based on the last recorded date with an assessment documenting absence of progressive disease.

The primary endpoint will be analyzed using a stratified log rank test, with ECOG PS 0 (Yes or No); presence of liver metastasis (Yes or No); elevated (1.5×ULN) CA 19-9 levels (Yes or No) as stratification factors. The effect of treatment will be estimated by the HR (anakinra versus placebo) together with its corresponding 95% confidence interval (CI) and p-value.

Key secondary objectives and endpoints: Secondary objectives and endpoints include: 1.To evaluate overall survival in patients with mPDAC receiving treatment with anakinra versus placebo, as add-on to GNP (overall survival is defined as the time from the date of randomization until death due to any cause); 2. To evaluate the effect of anakinra versus placebo, as add-on to GNP chemotherapy on different aspects of QoL in patients with mPDAC (EORTC QLQ-C30 domain scores will be used); 3. To evaluate early onset of efficacy of anakinra versus placebo, as add-on to GNP chemotherapy in the treatment of mPDAC (Carbohydrate antigen 19-9 (CA 19-9) response rate, Objective response rate by investigator assessment in accordance with RECIST 1.1. All responses will be confirmed 8 weeks after onset of response, and time to treatment failure, defined as the time from the start of GNP chemotherapy treatment to the date of discontinuation hereof for any reason); 4. To evaluate safety of anakinra as add-on to GNP chemotherapy (Adverse events, vital signs and laboratory safety assessments, including severity and duration of neutropenia; Proportion of patients receiving GNP chemotherapy according to protocol; GNP cycles where patients receive G-CSF.). Additionally, the exploratory objective of the study is to explore the effect of anakinra as add-on to GNP chemotherapy on inflammation markers.

The analysis of key secondary endpoints may be performed according to be following:

The key secondary endpoint overall survival is defined as the time from the date of randomization until death due to any cause. Any subject not known to have died at the time of analysis will be censored based on the last recorded date on which the subject was known to be alive.

The key secondary endpoints time to definitive deterioration 10 points) in each of the EORTC QLQ-C30 domain scores: fatigue, pain and loss of appetite may be analyzed using a stratified log rank test, with the same stratification factors as for the primary endpoint. The effect of treatment may be estimated by the HR (anakinra versus placebo) together with its corresponding confidence interval (CI) and p-value.

A multiple testing procedure, combining fixed sequence testing and Hochberg procedure, may be used to ensure an overall 1-sided significance level of 0.025 for the key secondary endpoints. A fixed sequential testing may be applied for the OS, the PFS and the three key QoL secondary endpoints: fatigue, pain and loss of appetite, i.e. the PFS will only be tested if the null hypothesis related to the OS is rejected and the QoL endpoints will only be tested if the null hypothesis related to the PFS is rejected. The Hochberg procedure may then be applied for the three key QoL secondary endpoints.

It is expected that administration of anakinra together with the chemotherapy as described above will result in a significant improvement of PDAC patient outcomes, such as stage IV PDAC patient outcomes, in comparison with the control group. Significant improvements are expected in one or several of the primary endpoint progression free survival and the key secondary endpoints overall survival listed above. The inventive treatment is expected to be tolerated well and fulfill safety requirements.

### ITEMIZED LIST OF EMBODIMENTS

1. Anakinra for use in the treatment of pancreatic ductal adenocarcinoma (PDAC), wherein said treatment comprises administration of a daily dose of at least approximately 200 mg, such as approximately 200 mg or at least 200 mg, anakinra to a patient in need thereof.

2. Anakinra for use according to item 1, wherein said patient is undergoing a PDAC treatment regimen, such as a chemotherapy treatment regimen and/or a radiotherapy treatment regimen.

3. Anakinra for use according to item 1 or 2, wherein said patient is undergoing a chemotherapy treament regimen.

4. Anakinra for use according to any one of items 1-3, wherein said daily dose of at least approximately 200 mg, such as approximately 200 mg, anakinra is administered at more than one separate administration occasion per day.

5. Anakinra for use according to any one of items 1-4, wherein said daily dose of at least approximately 200 mg, such as approximately 200 mg, anakinra is administered at least two separate administration occasions per day, such as at two separate administration occasions per day.

6. Anakinra for use according to any one of items 1-5, wherein said daily dose of at least approximately 200 mg, such as approximately 200 mg, anakinra administered on at least two separate administration occasion per day is administered as two or more essentially equal doses per day.

7. Anakinra for use according to any one of items 1-6, wherein said daily dose of at least approximately 200 mg, such as approximately 200 mg, anakinra administered on at least two separate administration occasion per day is administered as two doses per day of at least approximately 100 mg anakinra each, such as two doses of approximately 100 mg each, such as two doses of 100 mg anakinra each.

8. Anakinra for use according to any one of items 5-7, wherein said at least two separate administration occasions per day are at least approximately 8 hours apart.

9. Anakinra for use according to any one of items 5-8, wherein said at least two separate administration occasions per day are approximately 8-16 hours apart, such as approximately 9-15 hours apart, such as approximately 10-14 hours apart, such as approximately 11-13 hours apart, such as approximately 12 hours apart.

10. Anakinra for use according to any one of items 5-9, wherein said at least two separate administration occasions per day are approximately 12 hours apart.

11. Anakinra for use according to any one of items 1-10, wherein said administration is subcutaneous administration or intravenous administration.

12. Anakinra for use according to item 11, wherein said administration is subcutaneous administration, such as subcutaneous self-administration.

13. Anakinra for use according to item 11, wherein said administration is intravenous administration.

14. Anakinra for use according to any one of items 1-13, wherein said PDAC is stage IV PDAC.

15. Anakinra for use according to any one of items 1-14, wherein said treatment is preventive treatment of stage IV PDAC.

16. Anakinra for use according to any one of items 2-15, wherein said chemotherapy treatment regimen comprises days with administration of at least one chemotherapeutic agent and days without administration of at least one chemotherapeutic agent, and wherein said anakinra is administered during days with and without said administration of at least one chemotherapeutic agent.

17. Anakinra for use according to any one of items 2-16, wherein the patient is undergoing a chemotherapy treatment regimen and wherein

anakinra is administered continuously independent of the PDAC stage and/or identity of the chemotherapeutic agent.

18. Anakinra for use according to any one of items 2-17, wherein the patient is undergoing a first line of chemotherapy treatment regimen.

19. Anakinra for use according to any one of items 2-17, wherein the patient is undergoing a second or subsequent line of chemotherapy treatment regimen.

20. Anakinra for use according to any one of items 1-19, wherein said anakinra administration is continued after said patient has completed one or several chemotherapy treatment regimen(s).

21. Anakinra for use according to any one of items 1-2 and 4-15, wherein said patient is chemotherapy resistant.

22. Anakinra for use according to any one of items 2-20, wherein said chemotherapy treatment regimen comprises administration of therapeutically effective dose of at least one agent selected for the group consisting of actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, nanoliposomal irinotecan, leucovorin, mechlorethamine, mercaptopurine, methotrexate, mitoxantrone, oxaliplatin, paclitaxel, nab-paclitaxel, pemetrexed, pembrolizumab, platinum-based antineoplastic agents, teniposide, tioguanine, topotecan, valrubicin, vemurafenib, vinblastine, vincristine and vindesine, such as the group consisting of cisplatin, fluorouracil, gemcitabine, irinotecan, nanoliposomal irinotecan, leucovorin, nab-paclitaxel, oxaliplatin, pembrolizumab;

such as the group consisting of leucovorin, gemcitabine, nab-paclitaxel, cisplatin, fluorouracil, oxaliplatin and irinotecan;

such as the group consisting of gemcitabine, nab-paclitaxel, cisplatin, fluorouracil, oxaliplatin and irinotecan.

23. Anakinra for use according item 22, wherein said chemotherapy treatment regimen is selected from the group consisting of regimens a), b), c), d), e) and f), such as the group consisting of regimens a) and b),

wherein

regimen a) is administration of leucovorin, fluorouracil, irinotecan and oxaplatin;

regimen b) is administration of gemcitabine and nab-paclitaxel;

regimen c) is administration of pembrolizumab;

regimen d) is administration of fluorouracil and oxaplatin;

regimen e) is administration of fluorouracil and irinotecan; and

regimen f) is administration of fluorouracil and nanoliposomal irinotecan.

24. Anakinra for use according item 22 or 23, wherein said regimen comprises administration of gemcitabine or nab-paclitaxel.

25. Anakinra for use according to any one of items 22-24, wherein said regimen comprises administration of gemcitabine and nab-paclitaxel.

26. Anakinra for use according to item 25, wherein said administration of gemcitabine and nab-paclitaxel is on days 1, 8 and 15 of each 28-day cycle.

27. Anakinra for use according to item 26, wherein said administration is intravenous administration of approximately 1000 mg/m²gemcitabine and approximately 125 mg/m² nab-paclitaxel.

28. Anakinra for use according item 22 or 23, wherein said regimen comprises administration of leucovorin, fluorouracil, irinotecan or oxaplatin.

29. Anakinra for use according any one of items 22, 23 or 28, wherein said regimen comprises administration of leucovorin, fluorouracil, irinotecan and oxaplatin.

30. Anakinra for use according to any one of items 1-29, wherein said PDAC is stage IV PDAC.

31. A method of treatment of pancreatic ductal adenocarcinoma (PDAC), wherein said treatment comprises administering as therapeutically effective dose of at least approximately 200 mg, such as approximately 200 mg or at least 200 mg, anakinra per day to a patient in need thereof.

32. Method of treatment of PDAC according to item 31, wherein wherein said patient is undergoing a PDAC treatment regimen, such as a chemotherapy treatment regimen and/or a radiotherapy treatment regimen.

33. Method of treatment of PDAC according to item 31 or 32, wherein said patient is undergoing a chemotherapy treament regimen.

34. Method of treatment of PDAC according to any one of items 31-33, wherein said daily dose of at least approximately 200 mg, such as approximately 200 mg, anakinra is administered at more than one separate administration occasion per day.

35. Method of treatment of PDAC use according to any one of items 31-34, wherein said daily dose of at least approximately 200 mg, such as approximately 200 mg, anakinra is administered on at least two separate administration occasions per day, such as on two separate administration occasions per day.

36. Method of treatment of PDAC according to any one of items 31-35, wherein said daily dose of at least approximately 200 mg anakinra administered on at least two separate administration occasion per day is administered as two or more essentially equal doses per day.

37. Method of treatment of PDAC according to any one of items 31-36, wherein said daily dose of at least approximately 200 mg anakinra administered on at least two separate administration occasion per day is administered as two doses per day of at least approximately 100 mg anakinra each, such as two doses of approximately 100 mg each, such as two doses of 100 mg anakinra each.

38. Method of treatment of PDAC according to any one of items 35-37, wherein said at least two separate administration occasions per day are at least approximately 8 hours apart.

39. Method of treatment of PDAC according to any one of items 35-38, wherein said two separate administration occasions per day are approximately 8-16 hours apart, such as approximately 9-15 hours apart, such as approximately 10-14 hours apart, such as approximately 11-13 hours apart, such as approximately 12 hours apart.

40. Method of treatment of PDAC according to any one of items 35-39, wherein said two separate administration occasions per day are approximately 12 hours apart.

41. Method of treatment of PDAC according to any one of items 31-40, wherein said administration is subcutaneous administration or intravenous administration.

42. Method of treatment of PDAC according to item 41, wherein said administration is subcutaneous administration, such as subcutaneous self-administration.

43. Method of treatment of PDAC according to item 41, wherein said administration is intravenous administration.

44. Method of treatment of PDAC according to any one of items 31-43, wherein said PDAC is stage IV PDAC.

45. Method of treatment of PDAC according to any one of items 31-44, wherein said treatment is preventive treatment of stage IV PDAC.

46. Method of treatment of PDAC according to any one of items 32-45, wherein said chemotherapy treatment regimen comprises days with administration of at least one chemotherapeutic agent and days without administration of at least one chemotherapeutic agent, and wherein said anakinra is administered during days with and without said administration of at least one chemotherapeutic agent.

47. Method of treatment of PDAC according to any one of items 32-46, wherein the patient is undergoing a chemotherapy treatment regimen and wherein anakinra is administered continuously independent of the PDAC stage and/or identity of the chemotherapeutic agent.

48. Method of treatment of PDAC according to any one of items 32-47, wherein the patient is undergoing a first line of chemotherapy treatment regimen.

49. Method of treatment of PDAC according to any one of items 32-47, wherein the patient is undergoing a second or subsequent line of chemotherapy treatment regimen.

50. Method of treatment of PDAC according to any one of items 31-49, wherein said anakinra administration is continued after said patient has completed a chemotherapy treatment regimen.

51. Method of treatment of PDAC according to any one of items 31-32 and 34-45, wherein said patient is chemotherapy resistant.

52. Method of treatment of PDAC according to any one of items 32-50, wherein said chemotherapy treatment regimen comprises administration of therapeutically effective dose of at least one agent selected for the group consisting of actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, nanoliposomal irinotecan, leucovorin, mechlorethamine, mercaptopurine, methotrexate, mitoxantrone, oxaliplatin, paclitaxel, nab-paclitaxel, pemetrexed, pembrolizumab, platinum-based antineoplastic agents, teniposide, tioguanine, topotecan, valrubicin, vemurafenib, vinblastine, vincristine and vindesine,

such as the group consisting of cisplatin, fluorouracil, gemcitabine, irinotecan, nanoliposomal irinotecan, leucovorin, nab-paclitaxel, oxaliplatin, pembrolizumab;

such as the group consisting of leucovorin, gemcitabine, nab-paclitaxel, cisplatin, fluorouracil, oxaliplatin and irinotecan;

such as the group consisting of gemcitabine, nab-paclitaxel, cisplatin, fluorouracil, oxaliplatin and irinotecan.

53. Method of treatment of PDAC according item 52, wherein said chemotherapy treatment regimen is selected from the group consisting of regimens a), b), c), d), e) and f), such as the group consisting of regimens a) and b),

wherein

regimen a) is administration of leucovorin, fluorouracil, irinotecan and oxaplatin;

regimen b) is administration of gemcitabine and nab-paclitaxel;

regimen c) is administration of pembrolizumab;

regimen d) is administration of fluorouracil and oxaplatin;

regimen e) is administration of fluorouracil and irinotecan; and

regimen f) is administration of fluorouracil and nanoliposomal irinotecan.

54. Method of treatment of PDAC according item 52 or 53, wherein said regimen comprises administration of gemcitabine or nab-paclitaxel.

55. Method of treatment of PDAC according to any one of items 52-54, wherein said regimen comprises administration of gemcitabine and nab-paclitaxel.

56. Method of treatment of PDAC according to item 55, wherein said administration of gemcitabine and nab-paclitaxel is on days 1, 8 and 15 of each 28-day cycle.

57. Method of treatment of PDAC according to item 56, wherein said administration is intravenous administration of approximately 1000 mg/m²gemcitabine and approximately 125 mg/m² nab-paclitaxel.

58. Method of treatment of PDAC according item 52 or 53, wherein said regimen comprises administration of leucovorin, fluorouracil, irinotecan or oxaplatin.

59. Method of treatment of PDAC according any one of items 52, 53 or 58, wherein said regimen comprises administration of leucovorin, fluorouracil, irinotecan and oxaplatin.

60. Method of treatment of PDAC according to any one of items 31-59, wherein said PDAC is stage IV PDAC.

61. Use of anakinra for the manufacture of a medicament for the treatment of PDAC, wherein said treatment comprises administration of a daily dose of at least approximately 200 mg, such as approximately 200 mg, of anakinra to a patient in need thereof, such as wherein said treatment comprises administration of two doses per day of approximately at least 100 mg anakinra each, such as approximately 100 mg each, such as 100 mg each, to a patient in need thereof.

62. The use of anakinra for the manufacture of a medicament for the treatment of PDAC according to item 61, wherein said patient is undergoing a chemotherapy treatment regimen, such as a chemotherapy treatment regimen as defined in any one of items 22-29.

63. The use of anakinra for the manufacture of a medicament for the treatment of PDAC according to item 61 or 62, wherein said PDAC is stage IV PDAC.

64. A pharmaceutical composition comprising anakinra for use according to any one of items 1-30 and at least one pharmaceutically acceptable excipient, diluent and/or carrier.

65. The pharmaceutical composition comprising anakinra for use according to item 64, wherein said use is an add-on therapy to a PDAC treatment regimen, such as a chemotherapy treatment regimen.

1-31. (canceled)

32. A method of treatment of pancreatic ductal adenocarcinoma (PDAC), the method comprising:
administering a therapeutically effective dose of at least approximately 200 mg of anakinra per day to a patient in need thereof.

33. The method of treatment of PDAC according to claim 32, wherein said patient is undergoing a PDAC treatment regimen.

34. The method of treatment of PDAC according to claim 32, wherein said patient is undergoing a PDAC treatment regimen and said regimen is a chemotherapy treatment regimen.

35. The method of treatment of PDAC according to claim 32, wherein a daily dose of at least approximately 200 mg of anakinra is administered at more than one separate administration occasion per day.

36. The method of treatment of PDAC according to claim 32, wherein a daily dose of at least approximately 200 mg of anakinra is administered as two doses per day of at least approximately 100 mg of anakinra each, administered at two separate administration occasions per day.

37. The method of treatment of PDAC according to claim 36, wherein said two separate administration occasions per day are at least approximately 8 hours apart.

38. The method of treatment of PDAC according to claim 36, wherein said two separate administration occasions per day are approximately 8-16 hours apart.

39. The method of treatment of PDAC according to claim 32, wherein said administration is subcutaneous administration or intravenous administration.

40. The method of treatment of PDAC according to claim 32, wherein said administration is subcutaneous administration.

41. The method of treatment of PDAC according to claim 32, wherein said PDAC is stage IV PDAC.

42. The method of treatment of PDAC according to claim 34, wherein said chemotherapy treatment regimen comprises days with administration of at least one chemotherapeutic agent and days without administration of the at least one chemotherapeutic agent, and wherein said anakinra is administered during days with and days without said administration of the at least one chemotherapeutic agent.

43. The method of treatment of PDAC according to claim 32, wherein said anakinra administration is continued after said patient has completed one or several chemotherapy treatment regimen(s).

44. The method of treatment of PDAC according to claim 32, wherein said patient is chemotherapy resistant.

45. The method of treatment of PDAC according claim 34, wherein said chemotherapy treatment regimen comprises administration of a therapeutically effective dose of at least one agent selected from the group consisting of actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemicitabine, hydroxyurea, idarubicin, imatinib, irinotecan, nanoliposomal irinotecan, leucovorin, mechlorethamine, mercaptopurine, methotrexate, mitoxantrone, oxaliplatin, paclitaxel, nab-paclitaxel, pemetrexed, pembrolizumab, a platinum-based antineoplastic agent, teniposide, tioguanine, topotecan, valrubicin, vemurafenib, vinblastine, vincristine, and vindesine.

46. The method of treatment of PDAC according claim 34, wherein said chemotherapy treatment regimen is selected from the group consisting of regimens a), b), c), d), e), and f),
wherein
regimen a) is administration of leucovorin, fluorouracil, irinotecan, and oxaplatin;
regimen b) is administration of gemcitabine and nab-paclitaxel;
regimen c) is administration of pembrolizumab;
regimen d) is administration of fluorouracil and oxaliplatin;
regimen e) is administration of fluorouracil and irinotecan; and
regimen f) is administration of fluorouracil and nanoliposomal irinotecan.

47. The method of treatment of PDAC according claim 45, wherein said chemotherapy treatment regimen comprises administration of gemcitabine and/or nab-paclitaxel.

48. The method of treatment of PDAC according to claim 45, wherein said chemotherapy treatment regimen comprises administration of gemcitabine and nab-paclitaxel.

49. The method of treatment of PDAC according to claim 48, wherein said administration of gemcitabine and nab-paclitaxel is on days 1, 8, and 15 of each 28-day cycle.

50. The method of treatment of PDAC according to claim 48, wherein said administration is intravenous administration of approximately 1,000 mg/m² gemcitabine and approximately 125 mg/m² nab-paclitaxel.

51. The method of treatment of PDAC according to claim 45, wherein said chemotherapy treatment regimen comprises administration of leucovorin, fluorouracil, irinotecan, and oxaliplatin.
